(19) United States
(12) Patent Application Publication
(10) Pub. No.: US 2024/0018083 A1
(43) Pub. Date: Jan. 18, 2024

(54) METHOD FOR ISOLATING CARBOXYLIC ACID FROM AN AQUEOUS SIDE STREAM WITH CO-PRODUCTION OF ALKALI METAL SALT

(71) Applicant: NOURYON CHEMICALS INTERNATIONAL B.V., Amsterdam (NL)

(72) Inventors: Martinus Catharinus TAMMER, Diepenveen (NL); Antonie DEN BRABER, Arnhem (NL)

(73) Assignee: NOURYON CHEMICALS INTERNATIONAL B.V., Amsterdam (NL)

(21) Appl. No.: 18/254,453

(22) PCT Filed: Nov. 25, 2021
(86) PCT No.: PCT/EP2021/083073

§ 371 Date: May 25, 2023

(30) Foreign Application Priority Data
Dec. 1, 2020 (EP) 20210921.1

(51) Int. Cl. C07C 51/44; C07C 51/41; C07C 51/43; C07C 51/02

(52) U.S. Cl. CPC C07C 51/44; C07C 51/412; C07C 51/43; C07C 51/02

(57) ABSTRACT

Method for isolating carboxylic acid from an aqueous alkali metal carboxylate-containing side stream, e.g., the aqueous side stream of an organic peroxide production process, with co-production of alkali metal salt, involving adding an acid, or an anhydride, ketene or acid salt, to the aqueous side stream, thereby forming carboxylic acid and alkali metal salt within the aqueous side stream, and separation of the carboxylic acid from the aqueous side stream.

### CROSS-REFERENCE TO RELATED APPLICATION

This application is a U.S. National-Stage entry under 35 U.S.C. § 371 based on International Application No. PCT/EP2021/083073, filed Nov. 25, 2021 which was published under PCT Article 21(2) and which claims priority to European Application No. 20210921.1, filed Dec. 1, 2020, which are all hereby incorporated in their entirety by reference.

### TECHNICAL FIELD

This present disclosure relates to a method for isolating carboxylic acid from an aqueous side stream, such as, an aqueous side stream of an organic peroxide production process, with the co-production of alkali metal salt.

### BACKGROUND

Diacyl peroxides and peroxyesters can be prepared by reacting an anhydride or acid chloride with alkaline solutions of hydro(gen)peroxide, as illustrated by the following reaction schemes:

2R—C(═O)—O—C(═O)—R+M₂O₂→R—C(═O)—O—O—C(═O)—R+2MOC(═O)—R

R—C(═O)—O—C(═O)—R+ROOH+MOH→R—C(═O)—O—O—R+MOC(═O)—R

2R—C(═O)—Cl+M₂O₂→R—C(═O)—O—O—C(═O)—R+2MCl

R—C(═O)—Cl+ROOH+MOH→R—C(═O)—O—O—R+MCl

In this reaction scheme, M is Na or K. In addition, M₂O₂ does not refer to a discrete product M₂O₂, but to an equilibrium comprising H₂O₂ and MOOH.

Acid chlorides are relatively expensive and generate chloride-containing water layers, which lead to waste waters with high salt concentration.

Anhydrides, on the other hand, are even more expensive than acid chlorides and the side stream of the process starting with anhydride contains a high organic load—i.e. a high Chemical Oxygen Demand (COD) value—due to the formed carboxylic acid salt, and is therefore economically and environmentally unattractive.

That would change if the carboxylic acid could be isolated from the aqueous side stream and be re-used; either in a peroxide production process, in another chemical process (e.g. the production of esters), or in any other application, e.g. as animal feed ingredient.

CN108423908 discloses a process to isolate 4-methylbenzoic acid from a bis(4-methylbenzoyl)peroxide production process waste stream by precipitation. However, this process only works for acids with low solubility in water. In addition, the precipitate can cause smearing of the equipment used.

For carboxylic acids that are water-soluble or do not sufficiently precipitate or otherwise separate from the aqueous side stream, isolation is not easy or straightforward.

### SUMMARY

This disclosure provides a method for isolating carboxylic acid from an aqueous side stream with co-production of alkali metal salt, the method comprising the steps of:
a) providing an aqueous side stream comprising at least about 0.1 wt % of an alkali metal carboxylate, the alkali metal carboxylate being dissolved or homogeneously admixed within the stream,
b) adding an acid, or an anhydride, ketene or acid salt, to the aqueous side stream, thereby providing an aqueous mixture comprising carboxylic acid and alkali metal salt, the aqueous mixture being an aqueous monophasic mixture or an aqueous multiphasic mixture, and
c1) thermally separating the monophasic or multiphasic aqueous mixture to separate the carboxylic acid from the aqueous mixture, thereby providing a first stream comprising the carboxylic acid and a second stream comprising the alkali metal salt, or
c2) adding an organic solvent to the monophasic aqueous mixture to extract the carboxylic acid from the aqueous mixture, thereby providing a first stream comprising the carboxylic acid and a second stream comprising the alkali metal salt, and
d) optionally concentrating the second stream comprising the alkali metal salt by removal of water from the second stream.

This disclosure also provides a method for isolating an alkali metal salt from an aqueous side stream, the method comprising the steps of:
a) providing an aqueous side stream comprising at least about 0.1 wt % of an alkali metal carboxylate, the alkali metal carboxylate being dissolved or homogeneously admixed within the stream,
b1) adding an acid, or an anhydride, ketene or acid salt, to the aqueous side stream, thereby providing an aqueous mixture comprising carboxylic acid and alkali metal salt, the aqueous mixture being an aqueous monophasic mixture,
c) separating the carboxylic acid from the aqueous monophasic mixture, thereby providing a first stream comprising the carboxylic acid and a second stream comprising the alkali metal salt, and
d) optionally concentrating the second stream comprising the alkali metal salt by removal of water from the second stream.

It is an object of the present disclosure to provide a method for isolating such carboxylic acids from an aqueous side stream and make it suitable for re-use. It is a further object of the present disclosure for the method to be environmentally friendly, and thus create minimal waste, where possible.

Another object of the present disclosure is to provide a method for isolating an alkali metal salt from an aqueous side stream.

### DETAILED DESCRIPTION

The following detailed description is merely exemplary in nature and is not intended to limit the present disclosure or the application and uses of the present disclosure. Furthermore, there is no intention to be bound by any theory presented in the preceding background of the present disclosure or the following detailed description. It is to be appreciated that all numerical values as provided herein, save for the actual examples, are approximate values with endpoints or particular values intended to be read as “about” or “approximately” the value as recited.

In a first aspect, these objects are achieved by a process comprising the following steps:
a) providing an aqueous side stream comprising at least 0.1 wt % of an alkali metal carboxylate, the alkali metal carboxylate being dissolved or homogeneously admixed within the stream,
b) adding an acid, an anhydride, a ketene, or an acid salt, to the aqueous side stream, thereby providing an aqueous mixture comprising carboxylic acid and alkali metal salt, the aqueous mixture being an aqueous monophasic mixture or an aqueous multiphasic mixture, and
c1) thermally separating, preferably distilling, the monophasic or multiphasic aqueous mixture to separate the carboxylic acid from the aqueous mixture, thereby providing a first stream comprising the carboxylic acid and a second stream comprising the alkali metal salt, or
c2) adding an organic solvent to the monophasic aqueous mixture to extract the carboxylic acid from the aqueous mixture, thereby providing a first stream comprising the carboxylic acid and a second stream comprising the alkali metal salt, and
d) optionally concentrating the second stream comprising the alkali metal salt by removal of water from the second stream.

In a second aspect, these objects are achieved by a process comprising the following steps:
a) providing an aqueous side stream comprising at least 0.1 wt % of an alkali metal carboxylate, the alkali metal carboxylate being dissolved or homogeneously admixed within the stream,
b1) adding an acid, an anhydride, a ketene or an acid salt, to the aqueous side stream, thereby providing an aqueous mixture comprising carboxylic acid and alkali metal salt, the aqueous mixture being an aqueous monophasic mixture,
c) separating the carboxylic acid from the aqueous monophasic mixture, thereby providing a first stream comprising the carboxylic acid and a second stream comprising the alkali metal salt, and
d) optionally concentrating the second stream comprising the alkali metal salt by removal of water from the second stream.

In step c), the carboxylic acid may be separated from the aqueous monophasic mixture by thermally separating (preferably distilling) the monophasic aqueous mixture or by adding an organic solvent to the monophasic aqueous mixture to extract the carboxylic acid from the aqueous mixture.

The aqueous side stream may be obtained from any source. Preferably, the aqueous side stream is obtained from an organic peroxide production process, for example, the production of diacyl peroxides and/or peroxyesters. The organic peroxide production process leading to said aqueous side stream may involve the use of an acid chloride or an anhydride as reactant, preferably an anhydride.

Diacyl peroxides can be symmetrical or asymmetrical. Examples of suitable symmetrical diacyl peroxides produced in the organic peroxide production process leading to said aqueous side stream are di-2-methylbutyryl peroxide, di-isovaleryl peroxide, di-n-valeryl peroxide, di-n-caproyl peroxide, di-isobutyryl peroxide, and di-n-butanoyl peroxide. Examples of suitable asymmetrical diacyl peroxides produced in the organic peroxide production process leading to said aqueous side stream are acetyl isobutanoyl peroxide, acetyl 3-methylbutanoyl peroxide, acetyl lauroyl peroxide, acetyl isononanoyl peroxide, acetyl heptanoyl peroxide, acetyl cyclohexylcarboxylic peroxide, acetyl 2-propylheptanoyl peroxide, and acetyl 2-ethylhexanoyl peroxide.

Examples of suitable peroxyesters produced in the organic peroxide production process leading to said aqueous side stream are tert-butylperoxy 2-ethylhexanoate, tert-amylperoxy 2-ethylhexanoate, tert-hexylperoxy 2-ethylhexanoate, 1,1,3,3-tetramethyl butyl-1-peroxy 2-ethylhexanoate, 1,1,3,3-tetramethylbutyl 1-peroxyneodecanoate, tert-butylperoxy neodecanoate, tert-amylperoxy neodecanoate, tert-hexylperoxy neodecanoate, 1,1,3,3-tetramethylbutyl 1-peroxyneoheptanoate, tert-butylperoxy neoheptanoate, tert-amylperoxy neoheptanoate, tert-hexylperoxy neoheptanoate, 1,1,3,3-tetramethylbutyl 1-peroxyneononanoate, tert-butylperoxy neononanoate, tert-amylperoxy neononanoate, tert-hexylperoxy neononanoate, tert-butylperoxy pivalate, tert-amylperoxy pivalate, tert-hexyl-peroxy pivalate, 1,1,3,3-tetramethyl butyl-1-peroxy pivalate, tert-butylperoxy 3,3,5-trimethylhexanoate, tert-amylperoxy 3,3,5-trimethylhexanoate, tert-hexylperoxy 3,3,5-trimethylhexanoate, 1,1,3,3-tetramethyl butyl-1-peroxy 3,3,5-trimethylhexanoate, tert-butylperoxy isobutyrate, tert-amylperoxy isobutyrate, tert-hexylperoxy isobutyrate, 1,1,3,3-tetramethyl butyl-1-peroxy isobutyrate, tert-butylperoxy n-butyrate, tert-amylperoxy n-butyrate, tert-hexylperoxy n-butyrate, tert-butylperoxy isovalerate, tert-amylperoxy isovalerate, tert-hexylperoxy isovalerate, 1,1,3,3-tetramethyl butyl-1-peroxy isovalerate, tert-butylperoxy n-valerate, tert-amylperoxy n-valerate, tert-hexylperoxy n-valerate, 1,1,3,3-tetramethyl butyl-1-peroxy n-butyrate, 1,1,3,3-tetramethyl butyl 1-peroxy m-chlorobenzoate, tert-butylperoxy m-chlorobenzoate, tert-amylperoxy m-chlorobenzoate, tert-hexylperoxy m-chlorobenzoate, 1,1,3,3-tetramethyl butyl 1-peroxy o-methylbenzoate, tert-butylperoxy o-methylbenzoate, tert-amylperoxy o-methylbenzoate, tert-hexylperoxy o-methylbenzoate, 1,1,3,3-tetramethyl butyl 1-butylperoxy phenylacetate, tert-butylperoxy phenylacetate, tert-amylperoxy phenylacetate, tert-hexylperoxy phenylacetate, tert-butylperoxy 2-chloroacetate, tert-butylperoxy cyclododecanoate, tert-butylperoxy n-butyrate, tert-butylperoxy 2-methylbutyrate, tert-amyl-peroxy 2-methylburyrate, 1,1-dimethyl-3-hydroxy butyl-1-peroxy neodecanoate, 1,1-dimethyl-3-hydroxy butyl-1-peroxy pivalate, 1,1-dimethyl-3-hydroxy butyl-1-peroxy 2-ethylhexanoate, 1,1-dimethyl-3-hydroxy butyl-1-peroxy 3,3,5-trimethylhexanoate, and 1,1-dimethyl-3-hydroxy butyl-1-peroxy isobutyrate.

Preferred peroxyesters produced in the organic peroxide production process leading to said aqueous side stream include tert-butylperoxy isobutyrate, tert-amylperoxy isobutyrate, 1,1,3,3-tetramethyl butyl-1-peroxy isobutyrate, tert-butylperoxy n-butyrate, tert-amylperoxy n-butyrate, 1,1,3,3-tetramethyl butyl-1-peroxy n-butyrate, tert-butylperoxy isovalerate, tert-amylperoxy isovalerate, tert-butylperoxy 2-methylbutyrate, tert-amylperoxy 2-methylburyrate, 1,1,3,3-tetramethyl butyl-1-peroxy isovalerate, tert-butylperoxy n-valerate, tert-amylperoxy n-valerate, and 1,1,3,3-tetramethyl butyl-1-peroxy n-valerate.

The aqueous side stream comprises at least 0.1 wt %, preferably at least 1 wt %, more preferably at least 3 wt %, more preferably at least 5 wt %, more preferably at least 10 wt %, even more preferably at least 20 wt %, and most preferably at least 25 wt % of an alkali metal carboxylate dissolved or homogeneously admixed therein. The alkali metal carboxylate concentration is preferably not more than 65 wt %, more preferably not more than 60 wt %, and most preferably not more than 50 wt %. Thus, the aqueous side stream preferably comprises within the range of from 3 wt % to 65 wt %, more preferably from 20 wt % to 60 wt %, most preferably from 25 wt % to 50 wt %, of an alkali metal carboxylate dissolved or homogeneously admixed therein. For example, the aqueous side stream may comprise within the range of from 3 wt % to 65 wt %, more preferably from 20 wt % to 60 wt %, most preferably from 25 wt % to 50 wt %, of a potassium carboxylate dissolved or homogeneously admixed therein, or the aqueous side stream may comprise within the range of from 3 wt % to 65 wt %, more preferably from 20 wt % to 60 wt %, most preferably from 25 wt % to 50 wt %, of a sodium carboxylate dissolved or homogeneously admixed therein.

The concentration of the alkali metal carboxylate dissolved or homogeneously admixed within the aqueous side stream can be increased before step a) and/or between step a) and step b) (or step b1)) of the herein disclosed methods by removal of water from the aqueous side stream, for example, by thermal separation, e.g., distillation.

The alkali metal carboxylate is dissolved or homogeneously admixed with said stream, meaning that the stream includes a single phase and is not, e.g., a suspension containing alkali metal carboxylate particles. From such a suspension, the carboxylic acid could be easily separated by, e.g., filtration of the alkali metal carboxylate. From the aqueous stream of the present disclosure, however, such easy separation is not possible, and more steps are required to isolate the carboxylic acid.

Preferably, the alkali metal carboxylate is a potassium or sodium carboxylate salt of isobutyric acid, n-butyric acid, propionic acid, pivalic acid, neodecanoic acid, neoheptanoic acid, isononanoic acid, 2-methylbutyric acid, cyclohexylcarboxylic acid, lauric acid, isovaleric acid, n-valeric acid, n-hexanoic acid, 2-ethylhexanoic acid, heptanoic acid, octanoic acid, nonanoic acid, decanoic acid, lauric acid or mixtures thereof. More preferred alkali metal carboxylates are sodium or potassium carboxylate salts of isobutyric acid, n-butyric acid, n-heptanoic acid, n-octanoic acid, pivalic acid, isononanoic acid, 2-methylbutyric acid, cyclohexylcarboxylic acid, isovaleric acid and n-valeric acid. Most preferably, the alkali metal carboxylate is selected from sodium isobutyrate, potassium isobutyrate or a mixture of sodium isobutyrate and potassium isobutyrate, and the isolated carboxylic acid is isobutyric acid. The methods disclosed herein are particularly suitable for isolating carboxylic acids from an aqueous side stream with co-production of alkali metal salt, wherein the carboxylic acids have a water solubility of at least 0.1 g/100 mL, preferably at least 0.5 g/100 mL, more preferably at least 1 g/100 mL, more preferably at least 2 g/100 mL, more preferably at least 3 g/100 mL, more preferably at least 4 g/100 mL, and most preferably at least 5 g/100 mL, in each case as measured at 20° C.

When obtained from an organic peroxide production process, the aqueous side stream will contain some peroxide residues, such as organic hydroperoxide, hydrogen peroxide, peroxyacid, diacyl peroxide, and/or peroxyester. The peroxide content of the aqueous side stream will generally be in the range 0.01-3 wt %. The side stream may further contain some residual peroxide decomposition products.

In order to successfully isolate, purify, and re-use the carboxylic acid, any residual peroxides should preferably be removed from the aqueous side stream. This is done by extraction and/or the addition of a reducing agent. In addition, heating of the side stream may be desired.

Examples of suitable reducing agents are sodium sulfite, sodium (poly)sulfide (Na₂Sₓ), sodium thiosulfate, and sodium metabisulfite.

In a preferred embodiment, reducing agent is added to the aqueous side stream of an organic peroxide production process, either during step b) (or step b1)) or, more preferably, before step b) (or step b1)).

The reducing agent will destroy hydrogen peroxide, organic hydroperoxides, and peroxy acids. In order to destroy any other peroxidic species, it may be desired to increase the temperature of the aqueous side stream with 10-80° C., preferably 10-50° C., and most preferably 10-30° C. This temperature increase can be performed before step b) (or step b1)) or during step b) (or step b1)). If performed during step b) (or step b1)), any heat that is liberated by the addition of the acid, or an anhydride, ketene or acid salt, may be used to achieve this temperature increase. It should be noted that the temperature of the aqueous side stream before heating or addition of the acid, or an anhydride, ketene or acid salt, is generally in the range 0-20° C., preferably 0-10° C., as peroxide production processes are often performed at low temperatures.

Extraction can be performed before or after step b) (or step b1)) and is preferably performed before step b) (or step b1)). Extraction can be performed with organic solvents, anhydrides, and mixtures of anhydride and solvent. The organic layer obtained by the extraction can optionally be recycled to the organic peroxide production process.

Examples of suitable solvents for the extraction are alkanes (e.g. isododecane, Spirdane® and Isopar® mineral oils), chloroalkanes, esters (e.g. ethyl acetate, methyl acetate, dimethylphthalate, ethylene glycol dibenzoate, cumene, dibutyl maleate, di-isononyl-1,2-cyclohexaendicarboxylate (DINCH), dioctyl terephthalate, or 2,2,4-trimethylpentanediol diisobutyrate (TXIB)), ethers, amides, and ketones.

Examples of suitable anhydrides for the extraction are anhydrides that were or can be used in the organic peroxide production process and include symmetrical and asymmetrical anhydrides. Examples of symmetrical anhydrides are acetic anhydride, propionic anhydride, n-butyric anhydride, isobutyric anhydride, pivalic anhydride, valeric anhydride, isovaleric anhydride, 2-methylbutyric anhydride, 2-methylpentanoic anhydride, 2-methylhexanoic anhydride, 2-methylheptanoic anhydride, 2-ethylbutyric anhydride, caproic anhydride, caprylic anhydride, isocaproic anhydride, n-heptanoic anhydride, nonanoic anhydride, isononanoic anhydride, 3,5,5-trimethylhexanoic anhydride, 2-propylheptanoic anhydride, decanoic anhydride, neodecanoic anhydride, undecanoic anhydride, neoheptanoic anhydride, lauric anhydride, tridecanoic anhydride, 2-ethylhexanoic anhydride, myristic anhydride, palmitic anhydride, stearic anhydride, phenylacetic anhydride, cyclohexanecarboxylic anhydride, 3-methyl-cyclopentanecarboxylic anhydride, and mixtures of two or more of the above-mentioned anhydrides. Preferred symmetrical anhydrides are n-butyric anhydride, isobutyric anhydride, pivalic anhydride, valeric anhydride, isovaleric anhydride, 2-methylbutyric anhydride, 2-methylpentanoic anhydride, 2-methylhexanoic anhydride, 2-methylheptanoic anhydride, 2-ethylbutyric anhydride, caproic anhydride. Most preferred are n-butyric anhydride, isobutyric anhydride, valeric anhydride, isovaleric anhydride, 2-methylbutyric anhydride, 2-methylpentanoic anhydride.

Examples of suitable mixtures of symmetrical anhydrides are the mixture of isobutyric anhydride and 2-methylbutyric anhydride, the mixture of isobutyric anhydride and 2-methylpentanoic anhydride, the mixture of 2-methylbutyric anhydride and isovaleric anhydride, and the mixture of 2-methylbutyric anhydride and valeric anhydride.

Asymmetrical anhydrides are usually available as a mixture of the asymmetrical and symmetrical anhydrides. This is because asymmetrical anhydrides are usually obtained by reacting a mixture of acids with, e.g., acetic anhydride. This leads to a mixture of anhydrides, including an asymmetrical and at least one symmetrical anhydride. Such mixtures of anhydrides can be used for the extraction. Examples of suitable asymmetrical anhydrides are isobutyric 2-methylbutyric anhydride, which is preferably present as admixture with isobutyric anhydride and 2-methylbutyric anhydride; isobutyric acetic anhydride, which is preferably present as admixture with isobutyric anhydride and acetic anhydride, 2-methylbutyric valeric anhydride which is preferably present as admixture with 2-methylbutyric anhydride and valeric anhydride; and butyric valeric anhydride, which is preferably present as admixture with butyric anhydride and valeric anhydride.

More preferred anhydrides for the extraction are isobutyric anhydride, 2-methylbutyric anhydride, 2-methylhexanoic anhydride, 2-propylheptanoic anhydride, n-nonanoic anhydride, isononanoic anhydride, cyclohexanecarboxylic anhydride, 2-ethylhexanoic anhydride, caprylic anhydride, n-valeric anhydride, isovaleric anhydride, caproic anhydride, and lauric anhydride. Most preferred are isononanoic anhydride and isobutyric anhydride.

In step b) (or step b1)), an acid (or an anhydride, ketene or acid salt) is added to the aqueous side stream. This addition leads to the formation of an aqueous mixture comprising carboxylic acid and alkali metal salt, which is an aqueous monophasic mixture (i.e., a solution) or an aqueous multiphasic mixture.

The term “multiphasic mixture” is used herein to mean a biphasic or a triphasic mixture. In particular, the multiphasic mixture may be a biphasic mixture with two liquid phases, a biphasic mixture with one liquid phase and one solid phase caused by precipitation of the alkali metal salt, or a triphasic mixture with two liquid phases and one solid phase caused by precipitation of the alkali metal salt. In other words, the addition in step b) (or step b1)) does not lead to precipitation of the carboxylic acid which could then be easily separated from the mixture by, e.g., filtration. Instead, from the aqueous mixture of the present disclosure, such easy separation is not possible, and more steps are required to isolate the carboxylic acid.

The terms “aqueous monophasic mixture”, “aqueous biphasic mixture” and “aqueous triphasic mixture” are used herein to mean, respectively, a mono-, di-, or triphasic mixture that contains water in at least one phase.

Suitable acids and acid salts for addition in step b) (or step b1)) include sulfuric acid (H₂SO₄), hydrochloric acid (HCl), sodium hydrogen sulfate (NaHSO₄), potassium hydrogen sulfate (KHSO₄), phosphoric acid (H₃PO₄), oxalic acid, citric acid, formic acid, acetic acid, benzoic acid, and combinations thereof. Accordingly, the alkali metal salt formed in step b) (or step b1)) may be selected from alkali metal sulfate, alkali metal chloride, alkali metal hydrogen sulfate, alkali metal phosphate, alkali metal hydrogen phosphate, alkali metal dihydrogen phosphate, alkali metal oxalate, alkali metal citrate, alkali metal formate, alkali metal acetate, alkali metal benzoate, and combinations thereof. Preferably, the acids and acid salts for addition in step b) (or step b1)) are acids and acid salts with a pKₐ below 5.

The aqueous mixture formed in step b) is monophasic or multiphasic depending on:
the concentration of the alkali metal carboxylate within the aqueous side stream;
the carboxylic acid from which the alkali metal carboxylate is formed;
the acid, or anhydride, ketene or acid salt, added to the aqueous side stream in step b), including its dilution; and
the temperature of the aqueous mixture provided in step b).

In general, a higher concentration of alkali metal carboxylate, a higher carboxylic acid, a stronger acid, a more concentrated acid and/or a lower temperature, will lead to the formation of a multiphasic mixture from addition step b). For example, when an aqueous side stream comprising 2.4 wt % sodium pentanoate is acidified with 96% H₂SO₄, a clear solution (i.e., monophasic mixture) is obtained at 25° C., but at 20° C. and/or at a higher sodium pentanoate concentration, a biphasic mixture with two liquid phases is obtained. Likewise, when an aqueous side stream comprising 10 wt % sodium isobutyrate is acidified with 96% H₂SO₄ a clear solution (i.e., monophasic mixture) is obtained at 25° C., but at 20° C. and/or at a higher sodium isobutyrate concentration, a biphasic mixture with two liquid phases is obtained. Further, when the temperature is considerably lower than 20° C., a triphasic mixture with two liquid phases and a solid phase of precipitated Na₂SO₄ can be obtained. When adding a weaker acid however, such as acetic acid, a clear solution (i.e. monophasic mixture) can be obtained from an aqueous side stream comprising 30 wt % sodium pentanoate at 20° C.

Preferably, the aqueous mixture formed in step b) is an aqueous monophasic mixture or an aqueous biphasic mixture with two liquid phases.

In step b1), the concentration of the alkali metal carboxylate, the carboxylic acid from which the alkali metal carboxylate is formed, the acid (or anhydride, ketene or acid salt) added to the aqueous mixture and the temperature of the aqueous mixture are selected in accordance with the above such that an aqueous monophasic mixture is formed.

The acid, when monoprotic, may be added to the aqueous side stream in a molar ratio of added acid to alkali metal carboxylate in the aqueous side stream of at least 0.5:1, preferably at least 0.8:1, and more preferably at least 0.9:1. The molar ratio of added monoprotic acid to alkali metal carboxylate in the aqueous side stream may be at most 10:1, preferably at most 5:1, and more preferably at most 3:1. In particular, the molar ratio of added monoprotic acid to alkali metal carboxylate in the aqueous side stream may be of from 0.5:1 to 10:1, preferably of from 0.8:1 to 5:1, and more preferably of from 0.9:1 to 3:1. When the monoprotic acid is a strong acid, that is, a monoprotic acid that completely dissociates in aqueous solution (Ka>1, pKa<1), e.g., hydrochloric acid, the monoprotic acid is most preferably added to the aqueous side stream in a molar ratio of added acid to alkali metal carboxylate in the aqueous side stream of from 0.95:1 to 1.2:1. When the monoprotic acid is a weak acid, that is, a monoprotic acid that does not completely dissociate in aqueous solution (Ka<1, pKa>1), e.g., acetic acid, the monoprotic acid is most preferably added to the aqueous side stream in a molar ratio of added acid to alkali metal carboxylate in the aqueous side stream of from 1:1 to 5:1, more preferably of from 1:1 to 3:1, and most preferably of from 1.1:1 to 3:1.

When the acid is diprotic or triprotic at a pH of 4 in water, the molar ratio of added acid to alkali metal carboxylate in the aqueous side stream is adapted for the relevant number of equivalents of the acid. In this case, the molar ratio of added diprotic acid to alkali metal carboxylate in the aqueous side stream may be of from 0.2:1 to 5:1, preferably of from 0.4:1 to 2.5:1, and more preferably of from 0.4:1 to 1.5:1. When the diprotic acid is a strong acid, e.g., sulfuric acid, the diprotic acid is most preferably added to the aqueous side stream in a molar ratio of added acid to alkali metal carboxylate in the aqueous side stream of from 0.2:1 to 5:1, preferably of from 0.4:1 to 2.5:1, and more preferably of from 0.4:1 to 1.5:1. When the acid is a triprotic acid at a pH of 4 in water, the molar ratio of added triprotic acid to alkali metal carboxylate in the aqueous side stream may be of from 0.1:1 to 3.4:1, preferably of from 0.2:1 to 1.7:1, and more preferably of from 0.3:1 to 1:1.

In case an anhydride, ketene or acid salt is added to the aqueous side stream, the anhydride/ketene/acid salt hydrolyses to the respective acid when contacted with the water of the aqueous side stream. In such case, the “added acid” in the above disclosed molar ratios refer to the molar ratio of acid formed from the added anhydride, ketene or acid salt to alkali metal carboxylate in the aqueous side stream.

Preferred acids for addition in step b) (or step b1)) are sulfuric acid, phosphoric acid and acetic acid. If sulfuric acid (H₂SO₄) is used, it is preferably added as a 90-96 wt % solution.

The most preferred acid for addition in step b) (or step b1)) is acetic acid.

When acetic acid is used in step b) (or step b1)), it can be added as acetic acid, acetic anhydride and/or ethenone to form an aqueous mixture comprising carboxylic acid, alkali metal carboxylate, acetic acid and alkali metal acetate. More particularly, acetic acid, acetic anhydride and/or ethenone may be added to the aqueous side stream to form an aqueous monophasic mixture. For example, in the case of an aqueous side stream comprising at least 0.1 wt % of sodium isobutyrate, addition of acetic acid (or acetic anhydride and/or ethenone) leads to the formation of a homogeneous (i.e. monophasic) equilibrium mixture comprising isobutyric acid, sodium isobutyrate, acetic acid and sodium acetate.

Addition of the acetic acid, acetic anhydride and/or ethenone may be performed to obtain a pH below 7, preferably to obtain a pH below 6. The resulting pH is preferably not lower than 4.

The acetic acid, acetic anhydride and/or ethenone may be added to the aqueous side stream in a molar ratio of added acetic acid to alkali metal carboxylate in the aqueous side stream of at least 0.5:1, preferably at least 0.9:1, more preferably at least 1:1, more preferably 1.05:1, and most preferably at least 1.15:1. The molar ratio of added acetic acid to alkali metal carboxylate in the aqueous side stream may be at most 10:1, preferably at most 5:1, and more preferably at most 3:1. In particular, the molar ratio of added acetic acid to alkali metal carboxylate in the aqueous side stream may be of from 0.5:1 to 10:1, preferably of from 0.9:1 to 5:1, more preferably of from 1:1 to 5:1, more preferably of from 1:1 to 3:1, and most preferably of from 1.1:1 to 3:1.

The acetic acid is most preferably added in acid form.

The acetic acid used in step b) (or step b1)) may be obtained from any source. In one embodiment, the acetic acid is a high purity acetic acid produced industrially by methanol carbonylation, acetaldehyde oxidation, methyl formate isomerization, syngas conversion, gas phase oxidation of ethylene and ethanol and/or bacterial fermentation. Alternatively, the acetic acid may be prepared as a side product of another reaction, such as, an anhydride production process. Preferably, the acetic acid is liberated as a side product during preparation of the anhydride used to make the peroxide that generated the alkali metal carboxylate within the aqueous side stream (see reaction scheme on page 1).

Impurities in the acetic acid, such as water, carboxylate, carboxylic acid, etc., which do not hinder the alkali metal acetate crystallization (when desired), are allowed. Impurities in the acetic acid that are volatile in a next step and end up in the separated carboxylic acid are not preferred, except in the case where the impurity can be collected as a top stream in a thermal separation (e.g. distillation) step, or where the impurity is not detrimental to the uses of the carboxylic acid. For instance, the presence of acetic anhydride, acetyl isobutyryl anhydride and isobutyric acid in acetic acid from an isobutyric anhydride facility, are compatible impurities if the carboxylate water layer contains sodium isobutyrate coming from a di-isobutyryl peroxide process.

In steps c1) and c2), the carboxylic acid is separated from the monophasic or multiphasic aqueous mixture. Separation can be performed by thermal separation, such as distillation, or by extraction.

In step c), the carboxylic acid may be separated from the monophasic aqueous mixture by thermal separation, such as distillation, or by extraction. The thermal separation (e.g., distillation) or extraction may be performed in the same manner as described hereinafter in relation to step c1) and c2).

Removal of the carboxylic acid from the monophasic or multiphasic aqueous mixture may be from 10 wt % to greater than 99 wt %, preferably from 70 wt % to greater than 99 wt %, more preferably from 90 wt % to greater than 99 wt %, more preferably from 95 wt % to greater than 99 wt %, and most preferably from 95 wt % to 99 wt %, based on the total amount of alkali metal carboxylate dissolved or homogeneously admixed within the aqueous side stream provided to step a). In the case that the concentration of the alkali metal carboxylate dissolved or homogeneously admixed within the aqueous side stream provided to step a) is increased before step b) (or step b1)) of the herein disclosed methods, i.e., by removal of water from the aqueous side stream, the removal of the carboxylic acid from the monophasic or multiphasic aqueous mixture is based on the total amount of alkali metal carboxylate dissolved or homogeneously admixed within the aqueous side stream after concentration.

In step c1), the monophasic or multiphasic aqueous mixture is thermally separated, e.g., distilled, to separate the carboxylic acid from the aqueous mixture, thereby providing a first stream comprising carboxylic acid and a second stream comprising the alkali metal salt. The first stream may be an aqueous stream comprising the carboxylic acid and water. The second stream may be an aqueous stream comprising water and the alkali metal salt.

The first stream may be a monophasic or biphasic mixture comprising carboxylic acid and water. When the first stream is a biphasic mixture, the mixture may give two layers with a high and low carboxylic acid content. For example, the biphasic mixture may comprise (i) an aqueous layer comprising water and a minor amount of carboxylic acid and (ii) an organic liquid phase comprising carboxylic acid and a minor amount of water. In this document a minor amount is defined as 0 to 40 wt % based on total weight, preferably less than 35 wt %, more preferably less than 30 wt %, more preferably less than 25 wt % and most preferably less than 20 wt %.

As is known in the art, in thermal separation, two or more components are separated based on their difference in relative volatility. The component with a higher volatility is selectively vaporized from a liquid mixture and subsequently condensed. Thermal separation can result in complete separation of the components present in the starting liquid mixture or partial separation resulting in concentration of selected components in the liquid mixture. Thermal separation can be done using reduced pressure or at elevated pressure. Separation performance of a thermal separation step can be enhanced by providing several theoretical equilibrium stages either by using trays, structured packing or random packing. A reflux can be used wherein part of the condensed vapours are returned in the thermal separation unit to enhance the separation performance. Examples of thermal separation processes include flash distillation, vacuum distillation, fractional distillation, short path distillation, azeotropic distillation, extractive distillation, reactive distillation, stripping and rectification.

Preferably, thermal separation is performed by distillation. Distillation can be performed in a distillation or a stripping column, or a flash distillation may be used. The distillation may be carried out using reduced or elevated pressures. Performance may be enhanced by providing several theoretical equilibrium stages, either by using trays, structured packing or random packing, or by using a reflux. As mentioned above, when the carboxylic acid contains low boiling organics, such as alkanes, alcohols, esters, ethers or ketones, these can be separated during distillation as a top stream. Energy for the distillation can be supplied by an external heating source or live steam.

In embodiments where the aqueous mixture formed in step b) is multiphasic, such as biphasic, the multiphasic mixture is not separated into an aqueous liquid phase and an organic liquid phase prior to the thermal separation (e.g., distillation) of step c1).

As an alternative to thermal separation, in step c2), an organic solvent can be added to the monophasic aqueous mixture to extract the carboxylic acid from the aqueous mixture, thereby providing a first stream comprising the carboxylic acid and a second stream comprising the alkali metal salt. The first stream may be an organic solvent stream comprising the carboxylic acid and the organic solvent. The second stream may be an aqueous stream comprising water and the alkali metal salt.

When the first stream is an organic solvent stream comprising the carboxylic acid and the organic solvent, the carboxylic acid may be separated from the organic solvent by a subsequent thermal separation step, e.g., distillation. Depending on the relative boiling points of the organic solvent and the carboxylic acid, this may be done by separation (e.g., distillation) of the carboxylic acid from the higher boiling organic solvent or separation (e.g., distillation) of the organic solvent from the higher boiling carboxylic acid.

Extraction can be performed in any suitable device, such as a reactor, centrifuge, or mixer-settler. Optionally, extraction is performed with a wash section (i.e., a fractional extraction design) to limit the amount of acetic acid extracted with the carboxylic acid.

Examples of suitable organic solvents for the extraction are alkanes containing more than five carbon atoms (e.g. isododecane, Spirdane® and Isopar® mineral oils), alkenes containing more than five carbon atoms, chloroalkanes, esters (e.g. ethyl acetate, methyl acetate, dimethylphthalate, ethylene glycol dibenzoate, dibutyl maleate, di-isononyl-1,2-cyclohexaendicarboxylate (DINCH), dioctyl terephthalate, or 2,2,4-trimethylpentanediol diisobutyrate (TXIB)), ethers, aromatic compounds (cumene), amides, and ketones. Preferred solvents for the extraction are alkanes containing more than five carbon atoms and esters.

The extracted carboxylic acid/solvent mixture may contain impurities, water and acetic acid. Depending on the relative boiling points of the solvent and the carboxylic acid, the impurities, water and acetic acid may be removed by thermal separation (e.g., distillation) followed by thermal separation (e.g., distillation) of the carboxylic acid from the higher boiling solvent. When the solvent is one of the lowest boiling components, the carboxylic acid can be obtained as a residue of the thermal separations (e.g., distillations).

Preferably, the carboxylic acid is separated from the aqueous mixture formed in step b) (or step b1)) by thermally separating, more preferably distilling, the monophasic or multiphasic aqueous mixture (i.e., by step c1)).

Irrespective of whether the carboxylic acid is separated by step c1) or step c2), a subsequent separation or distillation step is optionally performed to further purify the carboxylic acid from low boiling organics, such as alkanes, alcohols, esters, ethers or ketones.

The distillation may also serve to evaporate volatile impurities, including water, from the carboxylic acid and/or to distill the carboxylic acid from any impurities with a boiling point higher than that of the carboxylic acid.

The term “distillation” in this specification includes any form of distillation, including flash distillation, vacuum distillation, fractional distillation, short path distillation, azeotropic distillation, extractive distillation and reactive distillation.

Cooling and/or the addition of a concentrated salt solution, e.g. a 10-35 wt % NaCl, NaHSO₄, KHSO₄, Na₂SO₄, (NH₄)₂SO₄ or K₂SO₄ solution, can be used to separate water from carboxylic acids with fewer than five carbon atoms. The salt reduces the solubility of the carboxylic acid in the aqueous liquid phase (i.e., “salting out”). Cooling is preferably performed to improve the separation, and may use temperatures<20° C., more preferably <10° C., and most preferably <5° C. to lower the solubility of the carboxylic acid in the water layer. When a concentrated salt solution is added, separation will be seen at higher temperatures, for example, at 40° C. However, the addition of a salt solution is not preferred due to the waste salt stream it generates. Aqueous mixtures of higher carboxylic acids (i.e., those having five or more carbon atoms), may give rise to two layers with a high and low carboxylic acid content. The first layer can be further purified by thermal separation (e.g., distillation), a drying salt, membrane process or any other drying technique to obtain a dry product. The second layer can be recycled to step a) or step b) (or step b1)).

Water obtained during the drying process can optionally be reused in the process that the carboxylate-containing side stream came from, e.g., an organic peroxide production process, or can be recycled to step a) or step b) (or step b1)).

The water content of the obtained carboxylic acid is preferably below 2 wt %, more preferably below 1 wt %, even more preferably below 0.5 wt %, and most preferably below 0.1 wt %. This is especially preferred in case the carboxylic acid will be re-used in a peroxide production process, in the anhydride step. As explained above, a further thermal separation (e.g., distillation) of the carboxylic acid may be required to reach this water content.

Preferred carboxylic acids to be obtained by the process of the present disclosure include isobutyric acid, n-butyric acid, propionic acid, pivalic acid, neodecanoic acid, neoheptanoic acid, isononanoic acid, 2-methylbutyric acid, cyclohexylcarboxylic acid, lauric acid, isovaleric acid, n-valeric acid, n-hexanoic acid, 2-ethylhexanoic acid, heptanoic acid, 2-propylheptanoic acid, octanoic acid, nonanoic acid, decanoic acid, and lauric acid. More preferred carboxylic acids are isobutyric acid, n-butyric acid, n-heptanoic acid, n-octanoic acid, pivalic acid, isononanoic acid, 2-methylbutyric acid, cyclohexylcarboxylic acid, isovaleric acid, and n-valeric acid.

The carboxylic acid obtained from the process of the present disclosure can be re-used in another application. For example, it can be recycled to the process from which it originated (e.g., an organic peroxide production process), it can be used in the production of another organic peroxide, and it can be used to make esters (for instance ethyl esters) that find use as, e.g., solvent or fragrance, or in agricultural applications.

The carboxylic acid—or a salt thereof—can also be used in animal feed. For instance, butyric acid salts are known to improve gastrointestinal health in poultry and prevent microbial infections and ailments in poultry, pigs, fishes, and ruminants.

The herein disclosed methods may include an additional step d), in which the amount of alkali metal salt within the second stream of step c1) or c2) or c) is concentrated by removal of water from the second stream. By removal of water from the second stream, a concentrated alkali metal salt solution and/or alkali metal salt may be provided. This is particularly preferred when the alkali metal salt is an alkali metal acetate, an alkali metal hydrogen phosphate, an alkali metal dihydrogen phosphate, or an alkali metal formate.

When the alkali metal salt is an alkali metal acetate (e.g. potassium acetate and/or sodium acetate), the metal acetate can be concentrated to a commercially viable solution, such as potassium acetate 10-70% or sodium acetate 8-60% (especially sodium acetate 20-60%, for example, sodium acetate 25% or 30%), or a commercially viable solid, such as potassium acetate, sodium acetate, sodium acetate-3 aq, and mixtures of potassium acetate and sodium acetate. Preferably, the alkali metal acetate is concentrated to potassium acetate as a 50% solution, potassium acetate crystals, sodium acetate as a 30% solution, sodium acetate as a 25% solution, sodium acetate crystals or sodium acetate-3 aq crystals.

By concentrating the alkali metal salt into a commercially viable product, the process of the present disclosure creates minimal waste to be disposed of. Hence, the process can be run with minimal environmental impact. As will be appreciated, additional step d) is not required if the alkali metal salt within the second stream of step c1) or c2) or c) is already at a useful concentration.

When the alkali metal salt is concentrated to a solution rather than a solid, any residual peroxides that may be present in the aqueous side stream are preferably removed without using sodium sulfite, sodium sulfide, sodium thiosulfate, sodium dithionite, their potassium salts, or any other sulfate producing reducing agent as the reducing agent.

Water is preferably removed via evaporation by thermal separation, e.g., distillation. Water may also be removed by the addition of alcohol to the stream. In the case of the formation of crystals, water is removed to a content of from 20 to 60 wt %, preferably followed by cooling. The crystals can be removed by filtration, centrifugation, etc., and the mother liquor recycled. In a more preferred method, the alkali metal salt, e.g., alkali metal acetate, is concentrated to a slurry of crystals in the saturated solution and then the crystals are removed by filtration, centrifugation, etc., and the mother liquor recycled. In this step, impurities that do not evaporate and crystalize will build up in the mother liquor so a part of the mother liquor might need to be discarded. Washing of the crystals may be necessary to improve their purity and may be performed using an elutriation leg, a centrifuge or a wash column. A wash of the crystals may be performed with water or with a sodium salt solution, e.g., sodium acetate solution, that has a lower impurity level than the mother liquor. The crystals may subsequently be dried.

Crystallization of the alkali metal salt may be assisted by seeding.

Solidification of the alkali metal salt may also be performed by contacting with a cold surface.

In case of impurities with a low solubility in the alkali metal salt solution, a purification step can be needed. For instance, sodium sulfate can be precipitated from the solution by cooling and precipitating as Na₂SO₄·10 aq. When the alkali metal salt is an acetate, Na₂SO₄·10 aq may optionally be precipitated in the presence of acetic acid before alkali metal acetate crystals are formed.

In case of impurities with a higher solubility in water than the alkali metal salt, after crystallization and solid/liquid separation, a part of the mother liquor can optionally be removed to afford for an outlet of the impurity.

The alkali metal salt that is produced by the herein disclosed methods can be used in another application. For example, potassium acetate obtained in step c1) or c2) or c) or step d) can be used in de-icing applications. In addition, sodium acetate obtained in step c1) or c2) or c) or step d) can be used to neutralize sulfuric acid (waste) streams, for removal of calcium salts, as a photoresist while using aniline dyes, as pickling agent in chrome tanning, to impede vulcanization of chloroprene, in processing cotton for disposable cotton pads, in food, in feed, in heating pads, in concreate sealing, in cleaning agents or in leather tanning

To further increase the amount of alkali metal salt that is produced by the herein disclosed methods, an alkali metal hydroxide may be added to the monophasic or multiphasic aqueous mixture before step c1) or c2) or c) in an amount to convert at least part, preferably all, of any free acid to the corresponding alkali metal salt. Additionally, or alternatively, alkali metal hydroxide can be added to the second stream comprising the alkali metal salt after step c1) or c2) or c) in an amount sufficient to neutralize at least part, preferably all, of any excess acid within the stream. Preferably, the alkali metal hydroxide is sodium hydroxide when the alkali metal carboxylate is sodium carboxylate, and potassium hydroxide when the alkali metal carboxylate is potassium carboxylate.

A particular embodiment of the present disclosure relates to a method for isolating isobutyric acid from an aqueous side stream with co-production of sodium acetate, the method comprising the steps of:
a) providing an aqueous side stream comprising at least 0.1 wt %, such as from 3 wt % to 65 wt % or from 25 wt % to 45 wt % or from 25 wt % to 45 wt %, of sodium isobutyrate, the sodium isobutyrate being dissolved or homogeneously admixed within the stream,
b) adding acetic acid, acetic anhydride and/or ethenone to the aqueous side stream in a molar ratio of added acetic acid to sodium isobutyrate in the aqueous side stream of from 0.5:1 to 10:1 (or from 1:1 to 5:1 or from 1:1 to 3:1), thereby providing an aqueous monophasic mixture comprising isobutyric acid and sodium acetate,
c) separating the isobutyric acid from the aqueous monophasic mixture, preferably by (i) thermally separating, preferably distilling, the aqueous monophasic mixture to separate the isobutyric acid from the aqueous mixture or (ii) adding an organic solvent to the monophasic aqueous mixture to extract the isobutyric acid from the aqueous mixture, more preferably separating the isobutyric acid from the aqueous monophasic mixture by distilling the aqueous mixture to separate the isobutyric acid from the aqueous mixture, thereby providing a first stream comprising isobutyric acid and a second stream comprising sodium acetate, and
d) optionally concentrating the second stream comprising the sodium acetate by removal of water from the second stream,
optionally wherein the aqueous side stream results from an organic peroxide production process, and
optionally further comprising recycling at least part of the isobutyric acid isolated in step c) to the organic peroxide production process.

Another particular embodiment of the present disclosure relates to a method for isolating isobutyric acid from an aqueous side stream with co-production of potassium acetate, the method comprising the steps of:
a) providing an aqueous side stream comprising at least 0.1 wt %, such as from 3 wt % to 65 wt % or from 25 wt % to 45 wt % or from 25 wt % to 45 wt %, of potassium isobutyrate, the potassium isobutyrate being dissolved or homogeneously admixed within the stream,
b) adding acetic acid, acetic anhydride and/or ethenone to the aqueous side stream in a molar ratio of added acetic acid to potassium isobutyrate in the aqueous side stream of from 0.5:1 to 10:1 (or from 1:1 to 5:1 or from 1:1 to 3:1), thereby providing an aqueous monophasic mixture comprising isobutyric acid and potassium acetate,
c) separating the isobutyric acid from the aqueous monophasic mixture, preferably by (i) thermally separating, preferably distilling, the aqueous monophasic mixture to separate the isobutyric acid from the aqueous mixture or (ii) adding an organic solvent to the monophasic aqueous mixture to extract the isobutyric acid from the aqueous mixture, more preferably separating the isobutyric acid from the aqueous monophasic mixture by distilling the aqueous mixture to separate the isobutyric acid from the aqueous mixture, thereby providing a first stream comprising the isobutyric acid and a second stream comprising the potassium acetate, and
d) optionally concentrating the second stream comprising the potassium acetate by removal of water from the second stream,
optionally wherein the aqueous side stream results from an organic peroxide production process, and
optionally further comprising recycling at least part of the isobutyric acid isolated in step c) to the organic peroxide production process.

Another particular embodiment of the present disclosure relates to a method for isolating sodium acetate from an aqueous side stream, the method comprising the steps of:
a) providing an aqueous side stream comprising at least 0.1 wt %, such as from 3 wt % to 65 wt % or from 25 wt % to 45 wt % or from 25 wt % to 45 wt %, of sodium carboxylate (e.g. sodium isobutyrate), the sodium carboxylate being dissolved or homogeneously admixed within the stream,
b) adding acetic acid, acetic anhydride and/or ethenone to the aqueous side stream in a molar ratio of added acetic acid to sodium carboxylate in the aqueous side stream of from 0.5:1 to 10:1 (or from 1:1 to 5:1 or from 1:1 to 3:1), thereby providing an aqueous monophasic mixture comprising carboxylic acid (e.g. isobutyric acid) and sodium acetate, and
c) separating the carboxylic acid from the aqueous monophasic mixture, preferably by (i) thermally separating, preferably distilling, the aqueous monophasic mixture to separate the carboxylic acid from the aqueous mixture or (ii) adding an organic solvent to the monophasic aqueous mixture to extract the carboxylic acid from the aqueous mixture, more preferably separating the carboxylic acid from the aqueous monophasic mixture by distilling the aqueous mixture to separate the carboxylic acid from the aqueous mixture, thereby providing a first stream comprising the carboxylic acid and a second stream comprising the sodium acetate,
d) optionally concentrating the second stream comprising the sodium acetate by removal of water from the second stream,
optionally wherein the aqueous side stream results from an organic peroxide production process, and
optionally further comprising recycling at least part of the carboxylic acid isolated in step c) to the organic peroxide production process.

Another particular embodiment of the present disclosure relates to a method for isolating potassium acetate from an aqueous side stream, the method comprising the steps of:
a) providing an aqueous side stream comprising at least 0.1 wt %, such as from 3 wt % to 65 wt % or from 25 wt % to 45 wt % or from 25 wt % to 45 wt %, of potassium carboxylate (e.g. potassium isobutyrate), the potassium carboxylate being dissolved or homogeneously admixed within the stream,
b) adding acetic acid, acetic anhydride and/or ethenone to the aqueous side stream in a molar ratio of added acetic acid to potassium carboxylate in the aqueous side stream of from 0.5:1 to 10:1 (or from 1:1 to 5:1 or from 1:1 to 3:1), thereby providing an aqueous monophasic mixture comprising carboxylic acid (e.g. isobutyric acid) and potassium acetate, and
c) separating the carboxylic acid from the aqueous monophasic mixture, preferably by (i) thermally separating, preferably distilling, the aqueous monophasic mixture to separate the carboxylic acid from the aqueous mixture or (ii) adding an organic solvent to the monophasic aqueous mixture to extract the carboxylic acid from the aqueous mixture, more preferably separating the carboxylic acid from the aqueous monophasic mixture by distilling the aqueous mixture to separate the carboxylic acid from the aqueous mixture, thereby providing a first stream comprising the carboxylic acid and a second stream comprising the potassium acetate,
d) optionally concentrating the second stream comprising the potassium acetate by removal of water from the second stream,
optionally wherein the aqueous side stream results from an organic peroxide production process, and
optionally further comprising recycling at least part of the carboxylic acid isolated in step c) to the organic peroxide production process.

### EXAMPLES

### Example 1: Isolation of Isobutyric Acid and Sodium Acetate-3 Aq from an Aqueous Side Stream

In a first step, a 2 liter glass reactor equipped with a cooling/heating mantle, a pitch blade impeller, and a thermometer was filled with: 1000 g of an aqueous side stream containing 25 wt % sodium isobutyrate (2.27 moles) having a temperature of 25° C., 260 g acetic acid (4.33 moles) having a temperature of 25° C., and 497 g of recycled filtrate (in this case, the sodium acetate solution obtained after crystallization and filtration of the solid sodium acetate wet product), having a temperature of 15° C. The molar ratio of added acetic acid to sodium isobutyrate in the aqueous side stream was 1.9:1. The resulting reaction mixture had a pH of 4.5-5.5 and was held at 20-30° C.

In a second step, the reaction mixture was heated to a temperature of 104° C. and 601 g of an isobutyric acid (IBA) solution in water was distilled via a rectifying column (a 40-centimeter Vigreux). The temperature was increased to 116° C.

Once the IBA concentration was low, the reaction mixture was heated to a temperature of 120° C. in a third step, and 234 g water was distilled.

In a fourth step, 182 g of a 50% w/w NaOH solution was added to the reaction mixture, to obtain a pH of 6-8, and the mixture was cooled to 45° C.

In a fifth step, the reaction mixture was cooled slowly from 45° C. to 15° C. and 5 mg sodium acetate crystals were added to the reaction mixture as seeds for crystallization at 35° C. This resulted in the formation of large sodium acetate crystals.

In a sixth step, 649 g sodium acetate crystals were filtered out of the reaction mixture, via a G3 glass filter applying vacuum suction, and dried with 20° C. air with 50% relative humidity, resulting in crystals of sodium acetate-3 aq. Of the filtrate obtained, 5 g was disposed of as waste and 450 g was recycled to the first step.

In a seventh step, the distillate containing the IBA solution in water was cooled to 2° C., causing separation into two layers containing IBA-76% w/w and IBA-18% w/w, respectively. The IBA-76% was distilled at 80° C. under vacuum to give 106 g dry IBA and 49 g of the IBA azeotrope, IBA-28%. The IBA-18% was distilled at 80° C. under vacuum to give 148 g pure water and 297 g of the IBA azeotrope, IBA-28% w/w. The IBA-28% streams were recycled to the cooling to 2° C. step (i.e., step 7).

The water obtained from step 3 (distillate) and step 7 (bottom) were combined and recycled to a peroxide production process. The excess water can also be used to make an emulsion of the peroxide end product (e.g., with methanol or ethanol).

The dry IBA obtained in the step 7 was found to contain some acetic acid (approx. 1% w/w). This is not a problem for recycling the IBA to an acid anhydride production step (where acetic acid is formed), such as an acid anhydride production step in organic peroxide production process. However, for alternative commercial production, a rectifying column with more trays and reflux, will result in a very low acetic acid content.

### Example 2: Isolation of Isobutyric Acid and Potassium Acetate Solution from an Aqueous Side Stream

In a first step, a 2 liter glass reactor equipped with a cooling/heating mantle, a pitch blade impeller, and a thermometer was filled with: 1000 g of an aqueous side stream containing 29 wt % potassium isobutyrate (2.30 moles) having a temperature of 25° C. and 157 g acetic acid (2.62 moles) having a temperature of 25° C. The molar ratio of added acetic acid to sodium isobutyrate in the aqueous side stream was 1.14:1 (˜1:1). The resulting reaction mixture had a pH of 5.0-5.5 and was held at 20-30° C.

In a second step, the reaction mixture was heated to a temperature of 102° C. and 629 g of an isobutyric acid (IBA) solution in water was distilled via a rectifying column (a 40-centimeter Vigreux). The temperature was increased to 116° C.

In a third step, once the IBA concentration was low, 36.1 g of a 50% KOH solution was added to the reaction mixture, to obtain a pH of 6-8.

In a fourth step, the reaction mixture was heated again, and 189 g water was distilled. (Here, the third and fourth steps can also be performed in reverse order).

The solution obtained was cooled to room temperature. Obtained was 514 g potassium acetate 50% w/w solution containing 0.1% w/w sodium isobutyrate. (For lower amounts of sodium isobutyrate, a more effective distillation column/reflux can be used.)

The IBA containing distillate streams were treated as in Example 1 and the water of the fourth stream was recycled.

This Example shows that only a 14 molar % excess of acetic acid is sufficient to be able to remove isobutyric acid.

### Example 3: Isolation of Valeric Acid and Sodium Acetate Solution from an Aqueous Side Stream

In a first step, 16.8 g acetic acid (0.28 mol) was added to 103.5 g of an aqueous side stream obtained from a peroxide process comprising 29.3 wt % sodium valerate (0.24 mol) and having a pH of 9.5 and a temperature of 20° C. The resulting mixture was homogeneous (i.e. monophasic) with a pH of 5.1 (Knick pH meter with a Toledo Inlab pH electrode).

In a second step, 40 g of n-heptane was added to the homogenous mixture and the mixture was mixed intensively at 20° C. The resulting mixture was then allowed to separate into a n-heptane layer and an aqueous layer, and the aqueous layer was extracted again with 40 g of n-heptane.

In a third step, the combined n-heptane layers were distilled to remove volatile components under vacuum, starting at 415 mbar at a reboiler temperature of 72° C. and increasing to 20 mbar at a reboiler temperature of 82° C. 23.2 g valeric acid (0.22 mol) was obtained with a content of >99 wt %. The distillate was 2 phases of which the lower aqueous phase was discarded, and the upper organic phase was mainly the n-heptane solvent that can be reused for the extraction step above (i.e., second step).

In a fourth step, 9.6 g NaOH-25% was added to the 93.5 g of the aqueous layer after the extraction step, which had a pH of 5.7. The resulting aqueous mixture had a pH of 7.2. The aqueous mixture was then concentrated by evaporating volatiles and water in a Rotavapor to 63° C. at 105 mbar, resulting in 74.6 g of an aqueous stream with a sodium acetate content of 27.5% and a sodium valerate content of 3.3%.

The sodium valerate content in the aqueous stream can be lowered by applying a larger excess of acetic acid in the acidification step of the sodium valerate (i.e., first step), and/or by using larger amounts of n-heptane in the extraction steps (i.e., second step) and/or more extraction steps/a multistage countercurrent extraction.

### Example 4: Method for Isolating Valeric Acid and Sodium Sulfate-10 Aq from an Aqueous Side Stream

In a first step, 13.5 g sulfuric acid (0.13 mol) was added to 103.5 g of an aqueous side stream obtained from a peroxide process comprising 28.8 wt % sodium valerate (0.24 mol) and having a pH of 9.5 (Knick pH meter with a Toledo Inlab pH electrode) and a temperature of 20° C. The resulting mixture was a biphasic mixture with a pH=2.

In a second step, the biphasic mixture was distilled at atmospheric pressure with a rectifying column, to remove the valeric acid as a mixture with water. The aqueous layer of the condensate was returned to the distillation flask. When 27.0 g of a valeric acid containing organic layer (86.5% valeric acid, 0.23 mol) and 38.2 g of a water layer (3.3% valeric acid, 0.01 mol) was collected, the distillation was stopped

The residue of the distillation was 51.8 g of a sodium sulfate solution with a pH<1. To the residue was added 5.8 g sodium hydroxide solution (16%, 0.023 mol) to neutralize the solution to pH 7.9 (Knick pH meter with a Toledo Inlab pH electrode).

On cooling to 0° C., a solid (sodium sulfate 10 aq) precipitated and 39.9 g solids were recovered by filtration (G3 glass filter and vacuum). 17.0 g of liquid filtrate was recovered (filtrate contained <0.1% valeric acid and had a low valeric acid smell).

### Example 5: Method for Isolating Valeric Acid and Sodium Propionate Solution from an Aqueous Side Stream

In a first step, 20.4 g propionic anhydride (0.156 mol) was added to 103.5 g of an aqueous side stream obtained from a peroxide process comprising 28.8 wt % sodium valerate (0.24 mol) and having a pH of 9.5 (Knick pH meter with a Toledo Inlab pH electrode) and a temperature of 20° C. Upon heating, the mixture became clear/homogeneous.

In a second step, valeric acid was distilled at atmospheric pressure with a rectifying column to remove the valeric acid as a mixture with water and propionic acid. The condensate was cooled in ice and the aqueous layer that formed was returned to the distillation flask. The distillation was stopped once an organic layer no longer separated. 24.2 g organics were obtained (67.6% Valeric acid, 0.16 mol) as was 23.9 g of a water layer of the distillate (4.2% Valeric acid, 0.01 mol).

The residue of the distillation was 74.6 g of a homogeneous solution containing valeric acid and propionic acid partly as free acid and sodium salt. The residue contained 10% Valeric acid (sum of free acid+sodium salt, 0.073 mol) and 26% of propionic acid (sum of free acid+sodium salt, 0.26 mol) (quantities determined by H-NMR).

It should be understood that Example 5 is based upon a non-optimized set of reaction conditions and has been included for the purposes of demonstrating that the method works with anhydrides.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. A method for isolating carboxylic acid from an aqueous side stream with co-production of alkali metal salt, the method comprising the steps of:
a) providing an aqueous side stream comprising at least about 0.1 wt % of an alkali metal carboxylate, the alkali metal carboxylate being dissolved or homogeneously admixed within the stream,
b) adding an acid, or an anhydride, ketene or acid salt, to the aqueous side stream, thereby providing an aqueous mixture comprising carboxylic acid and alkali metal salt, the aqueous mixture being an aqueous monophasic mixture or an aqueous multiphasic mixture, and
c1) thermally separating the monophasic or multiphasic aqueous mixture to separate the carboxylic acid from the aqueous mixture, thereby providing a first stream comprising the carboxylic acid and a second stream comprising the alkali metal salt, or
c2) adding an organic solvent to the monophasic aqueous mixture to extract the carboxylic acid from the aqueous mixture, thereby providing a first stream comprising the carboxylic acid and a second stream comprising the alkali metal salt, and
d) optionally concentrating the second stream comprising the alkali metal salt by removal of water from the second stream.

2. A method for isolating an alkali metal salt from an aqueous side stream, the method comprising the steps of:
a) providing an aqueous side stream comprising at least about 0.1 wt % of an alkali metal carboxylate, the alkali metal carboxylate being dissolved or homogeneously admixed within the stream,
b1) adding an acid, or an anhydride, ketene or acid salt, to the aqueous side stream, thereby providing an aqueous mixture comprising carboxylic acid and alkali metal salt, the aqueous mixture being an aqueous monophasic mixture,
c) separating the carboxylic acid from the aqueous monophasic mixture, thereby providing a first stream comprising the carboxylic acid and a second stream comprising the alkali metal salt, and
d) optionally concentrating the second stream comprising the alkali metal salt by removal of water from the second stream.

3. A method according to claim 1, wherein the carboxylic acid is selected from isobutyric acid, n-butyric acid, propionic acid, pivalic acid, neodecanoic acid, neoheptanoic acid, isononanoic acid, 2-methylbutyric acid, cyclohexylcarboxylic acid, lauric acid, isovaleric acid, n-valeric acid, n-hexanoic acid, 2-ethylhexanoic acid, heptanoic acid, octanoic acid, nonanoic acid, decanoic acid, lauric acid and mixtures thereof.

4. A method according to claim 1, wherein the alkali metal carboxylate is a sodium or potassium carboxylate salt of the carboxylic acid.

5. A method according to claim 1, wherein the aqueous side stream results from an organic peroxide production process, and optionally residual peroxides present in the aqueous side stream are removed by (i) extraction before or after step b) or step b1) and/or (ii) the addition of a reducing agent, heat or irradiation to the aqueous side stream before or after step b) or step b1).

6. A method according to claim 1 wherein the aqueous side stream in step a) comprises at least from about 3 wt % of the alkali metal carboxylate.

7. A method according to claim 1, wherein in step b) or step b1), acetic acid, acetic anhydride and/or ethenone is added to the aqueous side stream.

8. A method according to claim 7, wherein the acetic acid, acetic anhydride and/or ethenone is added to the aqueous side stream in a molar ratio of added acetic acid to alkali metal carboxylate in the aqueous side stream of from about 0.5:1 to about 10:1.

9. A method accordingly to claim 7, wherein the addition in step b) or step b1) is performed using acetic acid that is obtained as a side product of an anhydride production process, and comprises the carboxylic acid to be isolated.

10. A method according to claim 7, wherein the alkali metal salt is an alkali metal acetate.

11. A method according to claim 10, wherein the alkali metal acetate provided in step c1) or c2) or c) or d) is selected from potassium acetate 50% w/w, potassium acetate crystals, sodium acetate 25% w/w, sodium acetate 30% w/w, sodium acetate crystals and sodium acetate-3 aq crystals.

12. A method according to claim 1, wherein an alkali metal hydroxide is added (i) to the aqueous mixture provided in step b) or step b1), and/or (ii) to the second stream provided in step c1) or c2) or step c), the alkali metal hydroxide being added in an amount sufficient to neutralize at least part of any excess acid within the mixture and/or stream.

13. A method according to claim 1, further comprising purifying the carboxylic acid of the first stream by thermal separation, a drying salt or a membrane process.

14. A method according to claim 1, further comprising purifying the alkali metal salt of the second stream by precipitation and/or washing.

15. A method as claimed in claim 1, further comprising one or more of the following steps:
recycling at least part of the carboxylic acid separated in step c1) or c2) or c) to an organic peroxide production process;
using the carboxylic acid separated in step c1) or c2) or c) to make esters;
using the carboxylic acid isolated in step c1) or c2) or c) in animal feed;
using the alkali metal salt obtained in step c1) or c2) or c) or d) in de-icing applications, the alkali metal salt being potassium acetate; and/or
using the alkali metal salt obtained in step c1) or c2) or c) or d) to neutralize sulfuric acid (waste) streams, for removal of calcium salts, as a photoresist while using aniline dyes, as pickling agent in chrome tanning, to impede vulcanization of chloroprene, in processing cotton for disposable cotton pads, in food, in feed, in heating pads, in concreate sealing, in cleaning agents or in leather tanning, the alkali metal salt being sodium acetate.

16. A method according to claim 1 wherein the aqueous side stream in step a) comprises at least from about 10 wt % of the alkali metal carboxylate.

17. A method according to claim 1 wherein the aqueous side stream in step a) comprises at least from about 25 wt % of the alkali metal carboxylate.

18. A method according to claim 7, wherein the acetic acid, acetic anhydride and/or ethenone is added to the aqueous side stream in a molar ratio of added acetic acid to alkali metal carboxylate in the aqueous side stream of from about 1:1 to about 5:1.

19. A method according to claim 7, wherein the acetic acid, acetic anhydride and/or ethenone is added to the aqueous side stream in a molar ratio of added acetic acid to alkali metal carboxylate in the aqueous side stream of from about 1.1:1 to about 3:1.
